(19) United States
(12) Patent Application Publication
(10) Pub. No.: US 2002/0182158 A1
(43) Pub. Date: Dec. 5, 2002

(54) Methods for modifying the appearance of a substrate

(76) Inventors: Kalliope Christophides-Lordi, Clark, NJ (US); Maria Ulrich, Wayne, NJ (US); Anthony Potin, Paris (FR); Veronique Chevalier, Villecresnes (FR); Melanie Quest, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/080,066

(22) Filed: Feb. 22, 2002

(30) Foreign Application Priority Data
Feb. 22, 2001 (FR) 01 02423

(51) Int. Cl. A61K 7/025

(52) U.S. Cl. 424/64

(57) ABSTRACT

A method for modifying the appearance of a substrate comprising applying to the substrate a composition comprising at least one copolymer comprising at least one vinyl pyrrolidone monomeric unit and at least one C₁₀-C₄₀ alkene monomeric unit, wherein the at least one copolymer is present in an amount effective to modify the appearance of the substrate.

The present invention, in an embodiment, provides a method for modifying the appearance of a substrate, such as skin and/or semi-mucous membranes, comprising applying to the substrate a composition, such as a cosmetic composition, comprising at least one copolymer comprising at least one vinyl pyrrolidone monomeric unit and at least one C₁₀-C₄₀ alkene monomeric unit, wherein the at least one copolymer is present in an amount effective to modify the appearance of the substrate.

The color of skin may be influenced by various factors, such as the quantity of melanin in the skin, the level of blood circulation through the skin, and the temperature of the skin. For example, skin may redden under the effect of high temperature due to an increase in arterial blood flow through the skin. Similarly, skin may become blue under the effect of low temperature due to a decrease in arterial blood flow and a higher oxygen consumption by the metabolism.

The effects of metabolism and of external factors, such as temperature, on the color of skin may be exacerbated in the region surrounding the eye as this skin may be very thin. The effects of metabolism and of external factors may, for example, be observed as dark circles under, above, and/or around the eye. These dark circles may be exacerbated by allergic phenomena. Further, where the ocular region is concave, modifications in the reflection of the light due to the also a well-known consequence of lack of sleep, and may be due to a stagnation of blood around the eye. See Oresajo C. et al., Cosmetics and Toiletries, 102, 29-34 (1987)).

Although the causes of dark circles have not yet been completely elucidated, three types of dark circles are distinguishable by clinical examination: blue dark circles, which may be due to a decrease in micro-circulation around the eye, which, in turn, may cause an elevation of the reduced hemoglobin level; brown dark circles, which may be due to an accumulation of melanin pigments, such as accumulation due to age and/or exposure to UV light; and red dark circles.

Regardless of their color, dark circles are generally considered unaesthetic such that efforts have been made to mask or even remove them. To this end, it has been proposed to treat dark circles resulting from a weakening of the blood vessel with plant extracts such as venotonics (such as butcher's broom or horse chestnut), with vitamins such as vitamins A, K, E, B5 or C, or with draining agents such as caffeine. These plant extracts, vitamins, and draining agents may improve blood circulation, may reduce the fragility of the capillaries, and/or may strengthen the blood vessels in order to avoid their rupture.

Further, as disclosed in EP 1 090 629, the combination of dextran sulphate and escin may make it possible to reduce the dilation of capillaries in the skin and may exhibit an anti-oedematous effect useful in the treatment of peri-ocular dark circles.

It has also been proposed to conceal the dark circles using an optical effect provided by silica, mica, titanium dioxide and the like, and to lighten the skin around the eye with the aid of depigmenting agents such as the extracts of skullcap, mulberry, liquorice or camomile.

It has now been discovered, surprisingly, that the incorporation of certain copolymers formed from at least one vinyl pyrrolidone monomer and at least one C₁₀-C₄₀ alkene monomer into a composition, such as a cosmetic composition, may reduce the appearance of, including even cause the disappearance of, dark circles around the eyes when applied to the skin surrounding the eyes.

Copolymers formed from at least one vinyl pyrrolidone monomer and at least one C₁₀-C₄₀ alkene monomer have previously been described as film-forming compounds which may confer water-resistance properties on anti-sun compositions (see e.g., WO 00/41672, WO 95/19161, WO 97/42933) or on baby creams (see e.g., WO 94/14413) or non-transfer properties on make-up compositions (see e.g., EP 0 997 139, WO 99/22710, WO 98/16196, WO 97/17057, EP 0 819 428). Such copolymers have also been described as mascara constituents. See e.g., EP 0 792 636, and U.S. Pat. No. 5,389,363.

These copolymers have been used to improve a physical property of cosmetic compositions, such as retention, transfer resistance and/or water resistance. Compositions containing these copolymers are more particularly, for example, lipsticks, foundations, mascaras, eyeliners, self-tanning agents, anti-sun products and mosquito repellents.

In contrast, to the knowledge of the inventors, it has never been suggested to use at least one copolymer formed from at least one vinyl pyrrolidone monomer and at least one C₁₀-C₄₀ alkene monomer to modify the appearance of the skin and/or the semi-mucous membranes.

This novel application of these copolymers has, in addition, allowed the inventors to envisage using them for novel purposes, such as in compositions for treating oily or combination skin or in compositions for lessening, including correcting the signs of skin ageing and/or the cutaneous signs of fatigue.

Thus, in an embodiment, the present invention provides a method for reducing, including eliminating, the appearance of dark circles around eyes comprising applying to skin surrounding the eyes a composition comprising at least one copolymer formed from at least one vinyl pyrrolidone monomer and at least one C₁₀-C₄₀ alkene monomer, wherein the at least one copolymer is present in an amount effective to reduce the appearance of the dark circles.

Similarly, the incorporation of at least one copolymer formed from at least one vinyl pyrrolidone monomer and at least one C₁₀-C₄₀ alkene monomer into a composition may attenuate, or even cause the disappearance of, cutaneous signs of aging and/or fatigue. Thus, in another embodiment, the present invention provides a method for reducing, including eliminating, the cutaneous signs of aging and/or fatigue comprising applying to skin a composition comprising at least one copolymer formed at least one vinyl pyrrolidone monomer and at least one C₁₀-C₄₀ alkene monomer, wherein the at least one copolymer is present in an amount effective to reduce the appearance of the cutaneous signs of aging and/or fatigue.

In addition to dark circles and cutaneous signs of aging and/or fatigue, another type of visual irregularity may be observed on the skin, namely, a localized oily appearance. For example, glints caused by an excess of sebum may be observed on the surface of the skin. Such an oily appearance may be considered to be unaesthetic. Accordingly, products that confer a matte effect on skin may be desirable. In particular, such products may be highly sought after by consumers with combination skin or oily skin, as well as for products intended for use in hot or humid climates.

Similarly, it may be desirable, for aesthetic reasons, to confer a matte effect on the lips. Thus, for example, products, such as lipsticks, having a creamy and smooth texture but having a reduced luster or gloss may be desired.

Conventional methods for providing a matte effect, such as for reducing the gloss or shininess of skin or lips, generally consist of using fillers such as talc, starch, mica, silica, nylon powders, polyethylene powders, poly-beta-alanine powders, and polymethyl(meth)acrylate powders. However, fillers of this type may have the drawback of giving the skin or the lips an unnatural powdery appearance which may even accentuate their defects. Furthermore, these compositions may cause drying of the skin or of the lips in the long term and may be difficult to smooth over these substrates. Their matte effect may also be of a short duration. Moreover, difficulties associated with the introduction of these powders or fillers into compositions comprising a viscous oil, such as lanolin, for example lip products, may also exist because the composition can become too thick and trap air during molding.

However, it has been discovered, surprisingly, that copolymers formed from at least one vinyl pyrrolidone monomer and at least one C₁₀-C₄₀ alkene monomer may make it possible to make the skin and/or the lips matte without exhibiting at least one of the aforementioned disadvantages.

Thus, in an embodiment, the present invention provides a method for producing a matte effect on a substrate to which a cosmetic composition is applied, by applying to the substrate a cosmetic composition comprising at least one copolymer formed from at least one vinyl pyrrolidone monomer and at least one C₁₀-C₄₀ alkene monomer, wherein the at least one copolymer is present in an amount effective to produce a matte effect on the substrate. In an embodiment, the substrate is skin, such as human skin. In another embodiment, the substrate is chosen from semi-mucous membranes, such as human semi-mucous membranes. In an embodiment, semi-mucous membranes are lips.

In another embodiment, the present invention provides a method for treating oily or combination skin comprising applying to the skin a cosmetic composition comprising at least one copolymer formed from at least one vinyl pyrrolidone monomer and at least one C₁₀-C₄₀ alkene monomer, wherein the at least one copolymer is present in an amount effective to reduce or eliminate the oily appearance of oily or combination skin.

The present invention also provides, in an embodiment, a method for increasing the matte effect of a composition on a substrate comprising including in said composition at least one copolymer formed from at least one vinyl pyrrolidone monomer and at least one C₁₀-C₄₀ alkene monomer, wherein the at least one copolymer is present in an amount effective to increase the matte effect of the composition on the substrate.

In another embodiment, the present invention provides a method for reducing, including eliminating, the appearance of at least one of wrinkles, fine lines, and pores of a substrate comprising applying to the substrate a composition comprising at least one copolymer formed from at least one vinyl pyrrolidone monomer and at least one C₁₀-C₄₀ alkene monomer, wherein the at least one copolymer is present in an amount effective to reduce the appearance of at least one of wrinkles, fine lines, and pores of the substrate. In an embodiment, the substrate is skin, such as human skin. In another embodiment, the substrate is chosen from semi-mucous membranes, such as human semi-mucous membranes. In an embodiment, semi-mucous membranes are lips.

Certain terms used herein are defined below:

“At least one,” as used herein, refers to one or more and thus includes individual components as well as mixtures/combinations.

“Alkene,” as used herein, refers to substituted linear alkenes, unsubstituted linear alkenes, substituted branched alkenes, unsubstituted branched alkenes, substituted cyclic alkenes, and unsubstituted cyclic alkenes.

“Substituted,” as used herein, means further comprising at least one substituent. Non-limiting examples of substituents include atoms, such as oxygen, nitrogen, and halogens, as well as functional groups, such as hydroxyl, ether groups, oxyalkylene groups, polyoxyalkylene groups, carboxylic acid groups, amine groups, and amide groups.

“Copolymer,” as used herein, refers to polymers formed from at least two different types of monomers.

A “monomeric unit,” as used herein, is a monomer after it has been incorporated into a polymer.

The phrase “formed from,” as used herein, is open ended and does not limit the components of the at least one copolymer to at least one vinyl pyrrolidone monomer and at least one C₁₀-C₄₀ alkene monomer. Furthermore, the phrase “formed from” does not limit the order of adding monomers to form the at least one copolymer.

The phrase “modifying the appearance” of a substrate, as used herein, means modifying, including eliminating, at least one property visible or tactile on the surface of the substrate, including undesired properties. For example, modifying the appearance of a substrate includes providing a matte effect to the substrate, providing uniformity of color to a substrate, concealing non-uniformly colored portions of the substrate, and concealing wrinkles, lines, and pores in a substrate.

The phrase “undesired property visible or tactile on the surface of a substrate,” as used herein, includes imperfections in the surface of a substrate (such as wrinkles, fine lines, and pores), irregularities in the color of a substrate (such as dark circles around the eyes and other discolorations in the skin or lips), oiliness or glossiness of a substrate (such as that observed on oily type skin, that observed on combination type skin, as well as localized areas of oiliness on skin, e.g., the forehead and the wings of the nose), as well as the glossiness of a product, such as a cosmetic product, on a substrate (such as the glossiness of a lip product on the lips).

“Conceal,” as used herein, means to reduce, including eliminate, the visibility of a visible property of at least a portion of a substrate, such as color and texture (such as wrinkles, lines, and pores). Whether a visible property such as color is concealed is determined by measuring the difference between the reflectance of the area with undesired color and the reflectance of bare skin adjacent to the area with undesired color using a spectroradiometer. See Example 2, infra.

“Matte,” as used herein, refers to a non-shiny, non-glossy finish. As used herein, the term matte may be used to describe the finish provided by a composition to a substrate, such as rendering oily or combination skin matte, as well as to describe the finish of a composition on a substrate, such a matte film of lipstick on lips. The degree of matteness of a composition is measured using a gonioreflectometer to measure the reflection, R, of a composition, wherein R is the ratio of the specular reflection to the diffuse reflection. See Example 4, infra. The degree of matteness of a composition on a substrate is also measured using a gloss meter to measure the gloss of a film in terms of % reflectance. See Example 6, infra.

“Substrate,” as used herein, includes skin and semi-mucous membranes, such as human skin and human semi-mucous membranes.

“Semi-mucous membranes,” as used herein, includes lips, such as human lips.

It is to be understood that both the foregoing general description and the following detailed description are exemplary and explanatory only and are not restrictive of the invention as claimed. Reference will now be made in detail to exemplary embodiments of the present invention.

### BRIEF DESCRIPTION OF THE DRAWINGS

FIG. 1 plots the difference in reflectance as a function of the wavelength over the visible spectrum for Formula 1.

FIG. 2 plots the difference in reflectance as a function of the wavelength over the visible spectrum for Formula 2.

FIG. 3 plots the difference in reflectance as a function of the wavelength over the visible spectrum for Formula 3.

As discussed above, the present invention provides a method for modifying the appearance of a substrate comprising applying to the substrate a composition comprising at least one copolymer comprising at least one vinyl pyrrolidone monomeric unit and at least one C₁₀-C₄₀ alkene monomeric unit, wherein the at least one copolymer is present in an amount effective to modify the appearance of the substrate.

The present invention provides, in an embodiment, a method for modifying the appearance of skin and/or of semi-mucous membranes, such as human skin and/or human semi-mucous membranes, comprising applying to the skin and/or the semi-mucous membranes at least one composition comprising at least one copolymer formed from at least one vinyl pyrrolidone monomer and at least one C₁₀-C₄₀ alkene monomer, wherein the at least one copolymer is present in an amount effective to modify the appearance of skin and/or semi-mucous membranes. In an embodiment, the appearance of the skin and/or of the semi-mucous membranes is modified by an optical effect.

A subject of the present invention is therefore the cosmetic use of at least one copolymer formed from at least one vinyl pyrrolidone monomer and at least one C₁₀-C₄₀ alkene monomer in a cosmetic composition comprising a physiologically acceptable medium, as an agent for modifying the appearance of a substrate, such as the skin and/or the semi-mucous membranes. The at least one copolymer may, for example, be used according to the invention to make skin and/or lips matte, to soften dark circles around eyes, and/or to conceal wrinkles, fine lines, and/or pores of a substrate, such as skin and lips.

According to the present invention, the at least one copolymer is formed from at least one vinyl pyrrolidone monomer and at least one C₁₀-C₄₀ alkene monomer. Non-limiting examples of at least one C₁₀-C₄₀ alkene include pentadecene, hexadecene, heptadecene, octadecene, nonadecene, eicosene, docosene, and triacontene.

Non-limiting examples of the at least one copolymer include PVP/hexadecene copolymers (CFTA name), PVP/eicosene copolymers, (CTFA name), and tricontanyl PVP copolymers (CTFA name). In one embodiment, the at least one copolymer is tricontanyl PVP.

Non-limiting examples of PVP/eicosene copolymers include those sold by ISP under the tradenames Antaron V-220 (a PVP/eicosene copolymer comprising from 20% to 28% by weight of vinyl pyrrolidone and having a mass-average molecular weight of 8,600) and Ganex V-220 and those sold by Induchem under the tradename Unimer U-15. Further, non-limiting examples of PVP/hexadecene copolymers include those sold by ISP under the tradenames Antaron V-216 and Ganex V-216 and those sold by Induchem under the tradename Unimer U-151. Non-limiting examples of tricontanyl PVP copolymers include those sold by ISP under the tradenames Antaron WP-660 and Ganex WP-660 and those sold by Induchem under the tradename Unimer U-6.

In an embodiment, the weight-average molecular mass of the at least one copolymer ranges from 5,000 to 30,000. In another embodiment, the weight-average molecular mass of the at least one copolymer ranges from 6,000 to 20,000.

According to the present invention, the at least one copolymer is present in the composition in an amount effective to obtain the desired effect. For example, the amount of the at least one copolymer effective for modifying the appearance of skin ranges from 0.1% to 20% by weight relative to the total weight of the composition comprising the at least one copolymer. In another embodiment, the amount of the at least one copolymer effective for modifying the appearance of skin ranges from 0.5% to 10% by weight relative to the total weight of the composition comprising the at least one copolymer. In yet another embodiment, the amount of the at least one copolymer effective for modifying the appearance of skin ranges from 0.5% to 5% by weight relative to the total weight of the composition comprising the at least one copolymer.

The amount of the at least one copolymer effective for modifying the appearance of semi-mucous membranes ranges up to 3% by weight relative to the total weight of the composition comprising the at least one copolymer. As used herein (i.e., throughout the specification), “up to” includes the end point. In another embodiment, the amount of the at least one copolymer effective for modifying the appearance of semi-mucous membranes ranges from 0.1% to 2% by weight relative to the total weight of the composition comprising the at least one copolymer. These concentration ranges may make it possible to obtain a composition for semi-mucous membranes, such as lips, which is comfortable, which does not cause drying, and/or which exhibits good retention for at least four hours.

The composition according to the present invention may be in the form of a fluid, a cream, an ointment, a milk, a lotion, a serum, a paste, a foam, or a solid. The composition may also be optionally applied to a substrate in the form of an aerosol or a patch. Further, the composition may optionally be colored. Accordingly, the composition of the present invention may be a care product and/or a make-up product for a substrate, such as skin or lips.

The composition of the present invention may further comprise at least one suitable adjuvant commonly used in the field concerned. Non-limiting examples of the at least one adjuvant include hydrophilic gelling agents, lipophilic gelling agents, hydrophilic active agents, lipophilic active agents, preservatives, antioxidants, vitamins, depigmenting agents, solvents, perfumes, fillers, screening agents, pigments, odor absorbers, and colorants.

In an embodiment, the at least one adjuvant is present in the composition in an amount ranging from 0.01% to 20% by weight relative to the total weight of the composition. The at least one adjuvant, depending on its nature, may be introduced into the fatty phase or into the aqueous phase.

Needless to say, the person skilled in the art will take care to select optional additional additives and the amount thereof such that at least one advantageous property of the composition according to the invention is not, or is not substantially, adversely affected by the addition(s) envisaged.

The composition according to the present invention may be provided in any form normally used in the cosmetic field. For example, the composition may be in the form of an oily, optionally gelled, solution, a oil-in-water emulsion, a water-in-oil emulsion, a triple emulsion (such as a water-in-oil-in-water emulsion and an oil-in-water-in-oil emulsion), an vesicular, ionic type dispersion (such as liposomes and oleosomes), a vesicular, nonionic type dispersion (such as niosomes), a dispersion of nanocapsules, or a dispersion of nanospheres. Further, for example, the composition may be an anhydrous composition.

In an embodiment, the present invention provides a method of treating oily and combination skin comprising applying to the skin a composition comprising at least one copolymer formed from at least one vinyl pyrrolidone monomer and at least one C₁₀-C₄₀ alkene monomer. In an embodiment, the composition is in the form of an oil-in-water emulsion. In an embodiment, the external aqueous phase of an oil-in-water emulsion provides a cooling effect.

In an embodiment, the present invention provides a method for modifying the appearance of semi-mucous membranes, such as lips, comprising applying to the semi-mucous membranes a composition comprising at least one copolymer formed from at least one vinyl pyrrolidone monomer and at least one C₁₀-C₄₀ alkene monomer. In an embodiment, the composition is a lip product. In another embodiment, the composition is an anhydrous lip product, such as a lipstick.

In an embodiment, the composition of the present invention is in the form of an emulsion. In an embodiment, the proportion of the fatty phase in the composition in the form of an emulsion ranges from 5% to 80% by weight relative to the total weight of the composition. In another embodiment, the proportion of the fatty phase in the composition in the form of an emulsion ranges from 5% to 50% by weight relative to the total weight of the composition.

In an embodiment, the composition according to the present invention composition further comprises at least one compound chosen from emulsifiers and coemulsifiers. Emulsifiers and coemulsifiers suitable for use in a composition according to the present invention which is in the form of an emulsion include those conventionally used in the field considered. In an embodiment, emulsifiers and coemulsifiers are present in the composition in an amount ranging from 0.3% to 30% by weight relative to the total weight of the composition, such as from 0.5% to 20%. In an embodiment, the composition according to the invention in the form of an emulsion does not contain an emulsifier.

In an embodiment, the composition according to the present invention composition further comprises at least one oil. Oils suitable for use in a composition according to the present invention include those conventionally used in the field considered. Non-limiting examples of the at least one oil include:

hydrocarbon oils of animal origin, such as perhydrosqualene;

hydrocarbon oils of plant origin, such as liquid triglycerides of fatty acids comprising from 4 to 10 carbon atoms and the liquid fraction of shea butter;

synthetic esters and ethers, such as fatty acid esters and fatty acid ethers, including oils of formula R¹COOR² and of formula R¹OR², wherein R¹ is chosen from residues of a fatty acid comprising from 8 to 29 carbon atoms, and R² is chosen from branched hydrocarbon chains comprising from 3 to 30 carbon atoms and unbranched hydrocarbon chains comprising from 3 to 30 carbon atoms, such as for example purcellin oil, isononyl isononanoate, isopropyl myristate, 2-hexylethyl palmitate, 2-octyidodecyl stearate, 2-octyldodecyl erucate, isostearyl isostearate; hydroxylated esters such as isostearyl lactate, octyl hydroxystearate, octyidodecyl hydroxystearate, diisostearyl malate, triisocetyl citrate, heptanoates of fatty alcohols, octanoates of fatty alcohols, and decanoates of fatty alcohols; polyesters such as propylene glycol dioctanoate, neopentylglycol diheptanoate, and diethylene glycol diisononanoate; and esters of pentaerythritol such as pentaerythrityl tetraisostearate;

linear hydrocarbons of inorganic origin, linear hydrocarbons of synthetic origin, branched hydrocarbons of inorganic origin, and branched hydrocarbons of synthetic origin, such as volatile paraffin oils, nonvolatile paraffin oils, and derivatives thereof, petroleum jelly, polydecenes, and hydrogenated polyisobutene such as parleam oil;

fatty alcohols comprising from 8 to 26 carbon atoms, such as cetyl alcohol, stearyl alcohol and their mixture (cetylstearyl alcohol), octyl dodecanol, 2-butyloctanol, 2-hexyldecanol, 2-undecylpentadecanol, oleyl alcohol, and linoleyl alcohol;

partially hydrocarbon-based and/or partially silicone-based fluorinated oils such as those described in JP-A-2-295912; and

silicone oils such as volatile polymethylsiloxanes (PDMS) having at least one chain chosen from linear silicone-containing chains and cyclic silicone-containing chains and which are liquid or pasty at room temperature, nonvolatile polymethylsiloxanes (PDMS) having at least one chain chosen from linear silicone-containing chains and cyclic silicone-containing chains and which are liquid or pasty at room temperature, such as cyclopolydimethylsiloxanes (cyclomethicones) such as cyclohexasiloxane; polydimethylsiloxanes comprising at least one group comprising from 2 to 24 carbon atoms chosen from alkyl groups, alkoxy groups, and phenyl groups, pendant and/or at the end of a silicone-containing chain; and phenylated silicones such as phenyltrimethicones, phenyldimethicones, phenyltrimethylsiloxydiphenyl siloxanes, diphenyidimethicones, diphenylmethylidiphenyl trisiloxanes, 2-phenylethyltrimethyl siloxysilicates, and polymethylphenyl siloxanes.

Non-limiting examples of emulsifiers and coemulsifiers include oil-in-water emulsifiers such as fatty acid and polyethylene glycol esters (such as PEG-100 stearate) and fatty acid and glycerine esters (such as glyceryl stearate), and water-in-oil emulsifiers such as oxyethylenated poly(methylcetyl)(dimethyl)methylsiloxane (such as ABIL WE09 sold by Degussa Goldschmidt) and the mixture of ethylene glycol acetyl stearate and glyceryl tristearate sold by Guardian under the tradename UNITWIX.

Non-limiting examples of hydrophilic gelling agents include carboxyvinyl polymers (carbomer), acrylic copolymers such as acrylate/alkyl acrylate copolymers, polyacrylamides, polysaccharides, natural gums and clays. Non-limiting examples of lipophilic gelling agents include modified clays such as bentones, metal salts of fatty acids, hydrophobic silica, and polyethylenes.

Non-limiting examples of fillers include, in addition to pigments, silica powder; talc; starch crosslinked with octenylsuccinic anhydride sold by National Starch under the tradename DRY FLO PLUS (28-1160); polyamide particles such as those sold by Atochem under the tradename ORAGSOL; polyethylene powders; microspheres based on acrylic copolymers, such as those microspheres of ethylene glycol dimethacrylate/lauryl methacrylate copolymer sold by Dow Corning under the tradename POLYTRAP; expanded powders such as hollow microspheres, such as the microspheres sold by Kemanord Plast under the tradename EXPANCEL and the microspheres sold by Matsumoto under the tradename MICROPEARL F 80 ED; silicone resin microbeads such as those sold by Toshiba Silicone under the tradename TOSPEARL; and mixtures thereof. In an embodiment, fillers are chosen from silica, mica, and titanium dioxide.

According to the present invention, fillers may be present in the composition in an amount ranging up to 20% by weight relative to the total weight of the composition, such as from 1% to 10% by weight.

In an embodiment, the composition of the present invention, such as a composition for treating cutaneous signs of aging and/or of fatigue, further comprises at least one compound chosen from venotonic plant extracts such as extracts of butcher's broom and extracts of horse chestnut; vitamins such as vitamins A, K, E, B5 and C; xanthine bases such as caffeine; fillers; and depigmenting agents such as extracts of skullcap, extracts of mulberry, extracts of liquorice, and extracts of camomile.

In an embodiment, the composition of the present invention, such as a composition for treating oily or combination skin, further comprises at least one active agent chosen from vitamin B3; vitamin B5; zinc salts such as zinc oxide and zinc gluconate; salicylic acid; salicylic acid derivatives such as 5-(n-octanoyl)salicylic acid; triclosan; capryloylglycine; clove extracts; octopirox; hexamidine; azelaic acid; and azelaic acid derivatives.

According to the present invention, the at least one active agent may be incorporated into spherules, such as ionic vesicules, nonionic vesicules, and nanoparticles (such as nanocapsules and nanospheres), for example, in the case of incompatibility or to stabilize the at least one active agent.

The composition of the present invention may further comprise UVA and/or UVB screening agents chosen from organic screening agents and inorganic screening agents, wherein the agents are optionally coated in order to make them hydrophobic.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

### EXAMPLES

### Example 1

### Oil-in-water Emulsion Concealer

| | AMOUNT |
|---|---|
| COMPONENTS | (wt %) |
| PHASE A | |
| Glycols | 7% |
| Preservatives | 0.5% |
| Caffeine | 0.2% |
| Sodium chloride | 0.27% |
| Water | qs 100% |
| PHASE B | |
| Cyclopentasiloxane | 5.85% |
| Hydrogenated polyisobutane | 2% |
| Carbomer | 0.3% |
| Dimethiconol (gum) | 0.15% |
| Stearyl heptanoate and stearyl caprylate | 4% |
| PHASE B2 | |
| Tricontanyl PVP copolymer (Unimer U-6 from | 3% |
| Induchem) | |
| PHASE C | |
| Poly(2-acrylamido-2-methylpropane sulphonic acid) | 1.2% |
| crosslinked and neutralized with aqueous ammonia | |
| PHASE D | |
| Triethanolamine | 0.4% |
| Water | 3% |
| PHASE E | |
| Sodium hyaluronate | 1% |

Procedure

Phase A was heated with stirring at 80° C. until solubilization was obtained and then cooled to 65° C. Phase B1 was heated at 65° C. Phase B2 was then solubilized in phase B1, with stirring, to form phase B. Phase B was then added to phase A for emulsification using a Moritz device. Phase C was sprinkled over the resultant mixture of phases A and B and dispersed using a Moritz device. Phase D was then added, with stirring, to the mixture of phases A, B, and C and the resultant mixture was cooled to room temperature. Phase E was then added, with stirring, after pre-solubilization of its constituents.

### Example 2

### Demonstration of Increased Concealer Effect

The three following compositions were compared:

Formula 1 (Comparative): a composition corresponding to the composition of Example 1 in which the tricontanyl PVP was replaced with 3% by weight of silica.

Formula 2 (Comparative): a composition corresponding to the composition of Example 1 in which the tricontanyl PVP was replaced with 8% by weight of a mixture of silica and zinc oxide, wherein the amount of silica in the mixture ranged from 40% to 60% by weight relative to the total weight of the mixture, and wherein the amount of zinc oxide in the mixture ranged from 60% to 40% by weight relative to the total weight of the mixture.

Formula 3 (Inventive): a composition corresponding to the composition of Example 1 containing 3% by weight of tricontanyl PVP.

Each of the three compositions above was applied under the eyes of subjects each having different types of dark under-eye circles: blue, red and brown.

The difference between the reflectance of the area of the skin with dark circles and the reflectance of bare skin adjacent to the area with dark circles was measured using a spectroradiometer (Spectrascan PR650 PHOTORESEARCH; Standard: SRS-3 PHOTORESEARCH; Source: Illuminant D65; Angle of observation: 10°.).

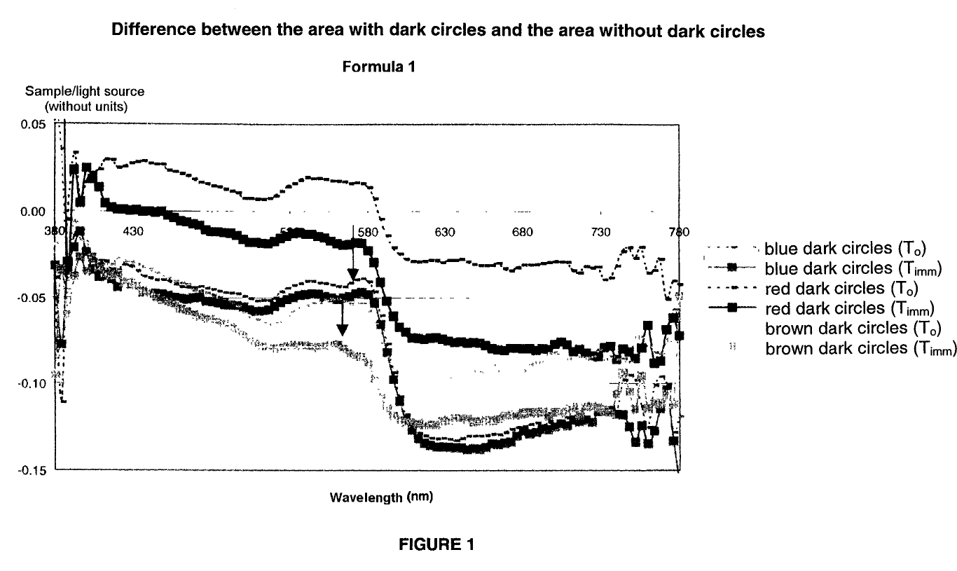

The difference in reflectance was plotted as a function of the wavelength over the whole visible spectrum. The results observed for Formulae 1, 2, and 3 are illustrated in FIGS. 1 to 3, respectively, wherein:

Tₒ corresponds to the difference in reflectance described above (skin with dark circles versus skin adjacent to this area) immediately preceding application of the composition, and

Tᵢₘₘ corresponds to the difference in reflectance described above immediately following application of the composition.

Reflectance corresponds to the behavior of the irradiated object towards the different wavelengths. The smaller the difference, after application of a composition, between the reflectance of the area with dark circles and the reflectance of bare skin adjacent to that area, the more similar the behaviors of the area with dark circles and the adjacent bare skin without dark circles towards the different wavelengths. In one embodiment, a difference in reflectance close to zero demonstrates a highly desired efficacy of the composition applied to the area with dark circles.

Accordingly, efficacy of the composition applied to the area with dark circles is tested by comparing (1) the difference in reflectance before application of the composition between the area with dark circles and the bare skin adjacent to that area, with (2) the difference in reflectance after application of the composition between the area with dark circles and the bare skin adjacent to the area with dark circles. Any reduction in difference (2) as compared to (1) demonstrates efficacy of the composition.

As shown in FIG. 1, an increase in the difference between the reflectance of the area with dark circles after application of Formula 1 and the reflectance of bare skin adjacent to the area with dark circles was observed for subjects with red and subjects with brown dark circles. An increase in the difference in reflectance demonstrates an intensification of the ring. However, for subjects with blue dark circles, no significant variation in the difference in reflectance was observed. This demonstrates a lack of efficacy of Formula 1 on blue dark circles.

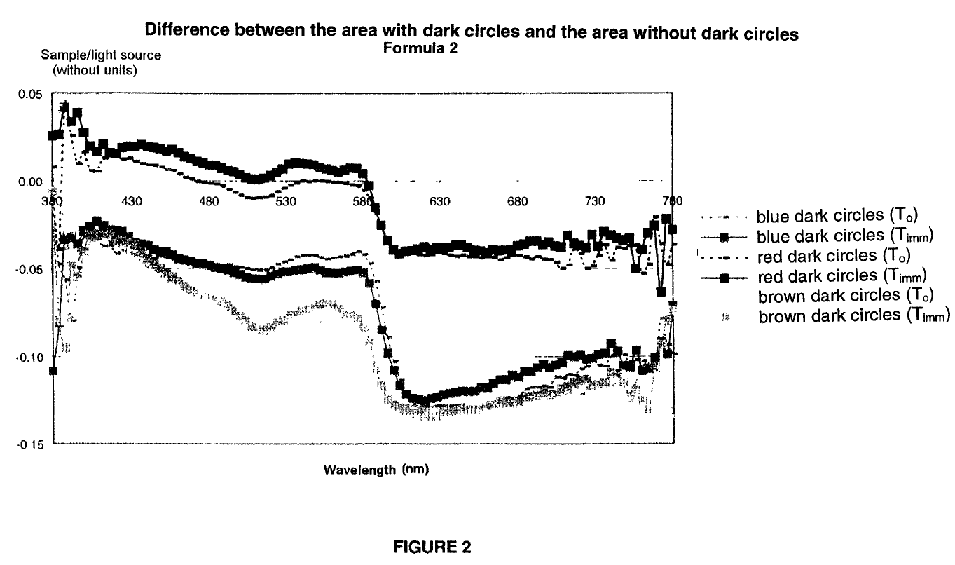

As shown in FIG. 2, no significant variation in the difference between the reflectance of the area with dark circles after application of Formula 2 and the reflectance of bare skin adjacent to the area with dark circles was observed for subjects with blue dark circles, subjects with red dark circles, or subjects with brown dark circles. This demonstrates a lack of desired efficacy of Formula 2 on blue, red, and brown dark circles.

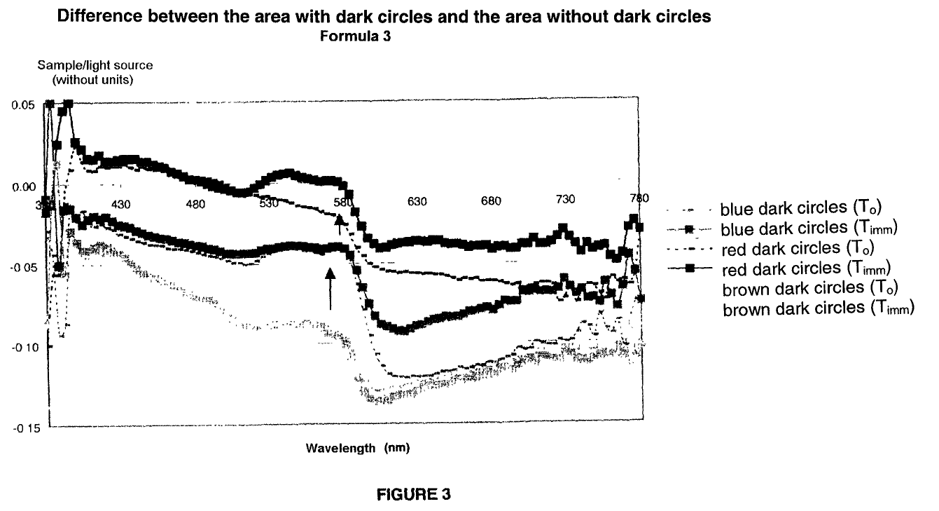

As shown in FIG. 3, a decrease in the difference between the reflectance of the area with dark circles after application of Formula 3 and the reflectance of bare skin adjacent to the area with dark circles was observed for subjects with red and subjects with blue dark circles. This demonstrates the efficacy of the inventive composition comprising vinylpyrrolidone/1-triacontene copolymer on the red and blue dark circles.

### Example 3

### Oil-in-water Emulsion

The following composition was prepared:

| | AMOUNT |
|---|---|
| COMPONENTS | wt % |
| OIL PHASE | |
| Stearyl alcohol | 1% |
| Dimyristyl tartrate/cetearyl alcohol/C₁₂-C₁₅-pareth-7/PPG- | 1.5% |
| 25 laureth-25 mixture (COSMACOL PSE from | |
| Enichem) | |
| Cyclohexadimethylsiloxane | 10% |
| Tricontanyl-PVP | 3% |
| AQUEOUS PHASE | |
| Glycerin | 5% |
| Ammonium polyacryldimethyltauramide (HOSTACERIN | 0.4% |
| AMPS from Hoechst) | |
| Aluminum starch octenyl succinate (DRY-FLO from | 3% |
| National Starch) | |
| Xanthan gum | 0.2% |
| Sodium hydroxide | 0.01% |
| Preservatives | 0.7% |
| Water | qs 100% |

The emulsion was prepared by adding the oil phase, with stirring, heated to 65° C., to the hot aqueous phase.

A mattifying composition was obtained which eliminates the glossiness of the skin.

### Example 4

### Demonstration of the Increased Matte Effect

The degree of matteness provided by the inventive composition of Example 3 comprising 3% tricontanyl PVP and that provided by a comparative composition according to Example 3 wherein the 3% of tricontanyl PVP in the fatty phase was replaced with 3% silica in the aqueous phase were measured.

The degree of matteness of each composition was measured by spreading 2 g/cm² of each composition on a rubber support and allowing the composition to dry. Using a gonioreflectometer, the reflection, R, of each of the dried samples was measured, wherein R is the ratio of the specular reflection to the diffuse reflection. (The angles of observation were 0° for the diffuse reflection and 30° for the specular reflection.) Accordingly, the higher the mattifying effect, the lower the value of R.

| Composition | R |
|---|---|
| Example 3 | 1.58 ± 0.03 |
| Comparative example | 1.92 ± 0.03 |

The results show that the degree of matteness of a composition comprising 3% (active substance) of tricontanyl PVP is much higher than that of a composition comprising 3% (active substance) of silica.

### Example 5

### Anhydrous Lipstick

The following anhydrous lipstick composition was prepared.

| | AMOUNT |
|---|---|
| COMPONENTS | (wt %) |
| Oil phase* | 65.53% |
| Vitamins (including antioxidants) | 0.5% |
| Waxes | 12.90% |
| Tricontanyl-PVP | 0.50% |
| Cholesterol | 0.10% |
| Silica dimethyl silylate | 2.00% |
| Pigments and fillers | 18.24% |
| 2-oleamido-1,3-octadecanediol | 0.05% |
| Perfume | 0.18% |

| | AMOUNT |
|---|---|
| COMPONENTS | (wt %) |
| Oil phase: | |
| Tricaprylin | 22.84% |
| Cetyl ethyl hexanoate | 22.92% |
| Cetyl acetate (and) acetylated lanolin | 3.05% |
| alcohol | |
| Isononyl isononanoate | 22.84% |
| Ricinus communis (castor-oil plant) seed oil | 29.96% |
| Stearalkonium hectorite | 1.07% |
| Propylene carbonate | 0.32% |

### Example 6

### Demonstration of the Increased Matte Effect

Three lipstick compositions were prepared: Composition A (inventive) according to Example 5 comprising 0.5% of tricontanyl-PVP, Composition B (comparative) according to Example 5 but not comprising tricontanyl-PVP, and Composition C (comparative) according to Example 5 wherein the tricontanyl-PVP was replaced with 0.5% of polybutene.

Each of the three lipsticks were applied to a laminated trial card and the gloss of the each of the resulting films was measured using a gloss meter (BYK Gardener, micro-PRI-gloss, Model 4525). The results presented below are expressed as % reflectance. (The angle of incidence was 60°.) The lower the % reflectance, the more matte (less glossy) the film. Seven to nine measurements were carried out on each composition and the mean was calculated. The results are shown in the table below.

| Composition A | Composition B | Composition C |
|---|---|---|
| (Inventive) | (Comparative) | (Comparative) |
| 3.2 | 7.0 | 6.7 |
| 3.8 | 11.5 | 10.2 |
| 6.5 | 11.6 | 12.5 |
| 4.9 | 9.5 | 13.4 |
| 3.1 | 7.4 | 16.1 |
| 5.2 | 8.8 | 17.1 |
| 5.1 | 8.5 | 14.2 |
| — | 9.6 | — |
| — | 9.5 | — |
| Mean: 4.54 | Mean: 9.26 | Mean: 12.89 |

The results show that the gloss of the inventive Composition A comprising 0.5% of tricontanyl-PVP was much lower and thus the matte effect was much increased compared to the two comparative compositions not comprising tricontanyl-PVP.

What is claimed is:

1. A method for modifying the appearance of a substrate comprising applying to the substrate a composition comprising at least one copolymer comprising at least one vinyl pyrrolidone monomeric unit and at least one C₁₀-C₄₀ alkene monomeric unit, wherein the at least one copolymer is present in an amount effective to modify the appearance of the substrate.

2. The method according to claim 1, wherein said substrate is chosen from skin.

3. The method according to claim 2, wherein said skin is human skin.

4. The method according to claim 1, wherein said substrate is chosen from semi-mucous membranes.

5. The method according to claim 4, wherein said semi-mucous membranes are human semi-mucous membranes.

6. The method according to claim 5, wherein said human semi-mucous membranes are human lips.

7. The method according to claim 1, wherein said at least one C₁₀-C₄₀ alkene monomeric unit is chosen from pentadecene, hexadecene, heptadecene, octadecene, nonadecene, eicosene, docosene, and triacontene.

8. The method according to claim 1, wherein said at least one copolymer is chosen from polyvinylpyrrolidone/hexadecene copolymers, polyvinylpyrrolidone/eicosene copolymers, and tricontanyl polyvinylpyrrolidone copolymers.

9. The method according to claim 1, wherein said at least one copolymer is tricontanyl polyvinylpyrrolidone.

10. The method according to claim 1, wherein said at least one copolymer has an weight-average molecular mass ranging from 5,000 to 30,000.

11. The method according to claim 10, wherein said at least one copolymer has an weight-average molecular mass ranging from 6,000 to 20,000.

12. The method according to claim 2, wherein said at least one copolymer is present in said composition in an amount ranging from 0.1% to 20% by weight relative to the total weight of said composition.

13. The method according to claim 12, wherein said at least one copolymer is present in said composition in an amount ranging from 0.5% to 10% by weight relative to the total weight of said composition.

14. The method according to claim 13, wherein said at least one copolymer is present in said composition in an amount ranging from 0.5% to 5% by weight relative to the total weight of said composition.

15. The method according to claim 4, wherein said at least one copolymer is present in said composition in an amount ranging up to 3% by weight relative to the total weight of said composition.

16. The method according to claim 15, wherein said at least one copolymer is present in said composition in an amount ranging from 0.1% to 2% by weight relative to the total weight of said composition.

17. The method according to claim 1, wherein said composition further comprises at least one compound chosen from venotonic plant extracts, vitamins, xanthine bases, fillers, and depigmenting agents.

18. The method according to claim 17, wherein said venotonic plant extracts are chosen from extracts of butcher's broom and extracts of horse chestnut.

19. The method according to claim 17, wherein said vitamins are chosen from vitamin A, vitamin K, vitamin E, vitamin B5, and vitamin C.

20. The method according to claim 17, wherein said xanthine bases are caffeine.

21. The method according to claim 17, wherein said depigmenting agents are chosen from extracts of skullcap, extracts of mulberry, extracts of liquorice, and extracts of camomile.

22. The method according to claim 1, wherein said composition further comprises at least one active agent chosen from vitamin B3, vitamin B5, zinc salts, salicylic acid, salicylic acid derivatives, triclosan, capryloylglycine, clove extracts, octopirox, hexamidine, azelaic acid, and azelaic acid derivatives.

23. The method according to claim 22, wherein said zinc salts are chosen from zinc oxide and zinc gluconate.

24. The method according to claim 22, wherein said salicylic acid derivatives are chosen from 5-(n-octanoyl)salicylic acid.

25. The method according to claim 1, wherein said composition is a cosmetic composition.

26. The method according to claim 1, wherein said composition is a colored composition.

27. The method according to claim 1, wherein said composition is an anhydrous composition.

28. The method according to claim 1, wherein said composition is in the form of a water-in-oil emulsion.

29. The method according to claim 1, wherein said composition is in the form of an oil-in-water emulsion.

30. The method according to claim 29, wherein the aqueous phase of said oil-in-water emulsion provides a cooling effect.

31. A method for reducing the appearance of dark circles around eyes comprising applying to skin surrounding the eyes a composition comprising at least one copolymer formed from at least one vinyl pyrrolidone monomer and at least one C₁₀-C₄₀ alkene monomer, wherein the at least one copolymer is present in an amount effective to reduce the appearance of the dark circles.

32. A method for reducing cutaneous signs of aging and/or fatigue comprising applying to skin a composition comprising at least one copolymer formed from at least one vinyl pyrrolidone monomer and at least one C₁₀-C₄₀ alkene monomer, wherein the at least one copolymer is present in an amount effective to reduce the appearance of the cutaneous signs of aging and/or fatigue.

33. A method for treating oily or combination skin comprising applying to the skin a cosmetic composition comprising at least one copolymer formed from at least one vinyl pyrrolidone monomer and at least one C₁₀-C₄₀ alkene monomer, wherein the at least one copolymer is present in an amount effective to reduce the oily appearance of oily or combination skin.

34. A method for reducing the appearance of at least one of wrinkles, fine lines, and pores of a substrate comprising applying to the substrate a composition comprising at least one copolymer formed from at least one vinyl pyrrolidone monomer and at least one C₁₀-C₄₀ alkene monomer, wherein the at least one copolymer is present in an amount effective to reduce the appearance of at least one of wrinkles, fine lines, and pores of the substrate.

35. A method for increasing the matte effect of a composition on a substrate comprising including in said composition at least one copolymer formed from at least one vinyl pyrrolidone monomer and at least one C₁₀-C₄₀ alkene monomer, wherein the at least one copolymer is present in an amount effective to increase the matte effect of the composition on the substrate.

36. A method for modifying the appearance of a substrate comprising applying to the substrate a composition comprising at least one copolymer formed from at least one vinyl pyrrolidone monomer and at least one C₁₀-C₄₀ alkene monomer, wherein the at least one copolymer is present in an amount effective to modify the appearance of the substrate.
